(19) United States
(12) Patent Application Publication
(10) Pub. No.: US 2013/0327342 A1
(43) Pub. Date: Dec. 12, 2013

(54) PERINEUM OVERLAY DEVICE

(71) Applicant: AMS Research Corporation, Minnetonka, MN (US)

(72) Inventors: Brian P. Watschke, Minneapolis, MN (US); Guangjian Wang, Falcon Heights, MN (US)

(73) Assignee: AMS Research Corporation, Minnetonka, MN (US)

(21) Appl. No.: 13/965,815

(22) Filed: Aug. 13, 2013

Related U.S. Application Data
(63) Continuation of application No. PCT/US2013/027914, filed on Feb. 28, 2013.
(60) Provisional application No. 61/604,034, filed on Feb. 28, 2012.

(51) Int. Cl. A61B 19/08

(52) U.S. Cl. CPC A61B 19/087
USPC 128/849

(57) ABSTRACT

A perineum overlay device for locating anatomical features within the perineum of a patient and/or needle insertion sites for targeting anatomical features within the perineum of a patient includes a section of flexible material and a plurality of markings printed on a first side of the section of the material. The section of flexible material is configured to conform and cover a portion of the perineum of a patient. The plurality of markings are each configured to identify a location of an anatomical feature within the perineum and/or a needle insertion site corresponding to a targeted anatomical feature within the perineum, when the section is positioned over the perineum of a patient.

### CROSS-REFERENCE TO RELATED APPLICATION

The present application is a continuation of and claims priority to International patent application Serial No. PCT/US2013/027914, filed Feb. 27, 2013, which claims the benefit of U.S. provisional patent application Ser. No. 61/604,034, filed Feb. 28, 2012, the content of which are hereby incorporated by reference in their entirety.

### FIELD

Embodiments of the invention are directed to a perineum overlay device for locating anatomical features within the perineum of a patient and/or needle insertion sites for targeting anatomical features within the perineum of a patient, and methods of using the device.

### BACKGROUND

Pelvic pain, dyspareunia and constipation are major stubborn pelvic health issues that currently do not have any good solutions. The etiology of these disorders is complex. One common mechanism of these seemingly disparate disorders is the pelvic floor muscle hypertonicity and/or spasm. Thus, therapies designed to alleviate pelvic floor muscle hypertonicity and/or spasm may prove to be effective in treating not just one but multiple pelvic disorders rooted from pelvic floor neuromuscular abnormalities. Pelvic floor muscle injection of drugs such as Botox, may represent a promising minimally invasive treatment for these disorders with potentially high therapeutic efficacies.

### SUMMARY

Embodiments of the invention are directed to a perineum overlay device for locating anatomical features within the perineum of a patient and/or needle insertion sites for targeting anatomical features within the perineum of a patient, and methods of using the device to treat a pelvic condition of a patient. In some embodiments, the device includes a section of flexible material and a plurality of markings printed on a first side of the section of the material. The section of flexible material is configured to conform and cover a portion of the perineum of a patient. The plurality of markings are configured to identify a location of an anatomical feature within the perineum and/or a needle insertion site corresponding to a targeted anatomical feature within the perineum, when the section is positioned over the perineum of a patient.

In some embodiments, the device includes an adhesive layer on a second side of the section that is opposite the first side. The adhesive assists in bonding the device over the perineum of the patient.

In some embodiments, the section of flexible material is configured to cover the vagina, the urethra, and/or the anus of a patient.

In some embodiments, the markings include at least one anatomical marking configured to identify a location of one or more muscles of the levator ani. In some embodiments, the muscles identified by the anatomical markings include the puborectalis, the pubococcygeus and/or the iliococcygeus. In some embodiments, the anatomical markings are configured to identify a location of one or more anatomical features of a patient. In some embodiments, the anatomical features include the vagina, the urethra, and/or the anus.

In some embodiments, the markings include at least one needle insertion site marking configured to identify a needle insertion site.

In some embodiments, the markings comprise graphical markings. In some embodiments, the markings comprise textual markings. In some embodiments, the markings comprise one or more openings in the section of flexible material.

In some embodiments, the device includes a needle stop attached to the first side of the section. The needle stop includes a cylindrical bore through which a needle may be inserted. In some embodiments, the needle stop is positioned at a needle insertion site. In some embodiments, the needle stop is positioned over one of the markings identifying a needle insertion site.

In some embodiments of the method, a perineum overlay device comprising a section of flexible material is positioned over the perineum of a patient. In some embodiments, alignment markings on a first side of the section are aligned with corresponding features on the perineum. A second side of the section is bonded to the perineum. A needle is inserted through a needle insertion site marking or an anatomical marking on the first side of the section and into tissue of the patient. A pelvic treatment is then performed using the needle.

In some embodiments, the alignment markings comprise at least one anatomical marking identifying an anatomical feature of the patient. In some embodiments, the anatomical features of the patient include the vagina, the urethra, and/or the anus. In some embodiments, the alignment of the alignment markings with corresponding features of the perineum comprises aligning at least one anatomical marking with the corresponding anatomical feature of the patient.

In some embodiments, the alignment markings comprise at least one opening. In some embodiments, the alignment of the alignment markings comprises aligning at least one opening to a mark applied to the epidermis of the perineum of the patient or an anatomical feature of the patient.

In some embodiments, the bonding of the second side of the section to the perineum comprises bonding an adhesive layer attached to the second side of the section of flexible material to epidermis of the perineum.

In some embodiments, the insertion of the needle through the needle insertion site marking or anatomical marking and into tissue of the patient comprises inserting a needle through a cylindrical bore of a needle stop attached to the first side of the section, and limiting a depth at which the needle is inserted into the tissue through engagement with the needle stop.

In some embodiments, the performance of a pelvic treatment using the needle comprises injecting a substance into the tissue, recording electromyogram signals, electrically stimulating the tissue, and/or implanting and electrode in the tissue.

This Summary is provided to introduce a selection of concepts in a simplified form that are further described below in the Detailed Description. This Summary is not indented to identify key features or essential features of the claimed subject matter, nor is it intended to be used as an aid in determining the scope of the claimed subject matter. The claimed subject matter is not limited to implementations that solve any or all disadvantages noted in the Background.

### BRIEF DESCRIPTION OF THE DRAWINGS

FIG. 1 is a top view of a perineum overlay device in accordance with embodiments of the invention.

FIG. 2 illustrates anatomy of the pelvic region.

FIG. 3 illustrates the placement of a perineum overlay device on the perineum of a patient, in accordance with embodiments of the invention.

FIG. 4 is a cross-sectional view of a portion of a perineum overlay device in accordance with embodiments of the invention overlaying the perineum of a patient.

FIG. 5 is a flowchart illustrating a method of treating a pelvic condition of a patient in accordance with embodiments of the invention.

### DETAILED DESCRIPTION OF ILLUSTRATIVE EMBODIMENTS

Embodiments of the invention are described more fully hereinafter with reference to the accompanying drawings. The various embodiments of the invention may, however, be embodied in many different forms and should not be construed as limited to the embodiments set forth herein. Rather, these embodiments are provided so that this disclosure will be thorough and complete, and will fully convey the scope of the invention to those skilled in the art. Elements that are identified using the same or similar reference characters refer to the same or similar elements.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be understood that, although the terms first, second, etc. may be used herein to describe various elements, these elements should not be limited by these terms. These terms are only used to distinguish one element from another. Thus, a first element could be termed a second element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

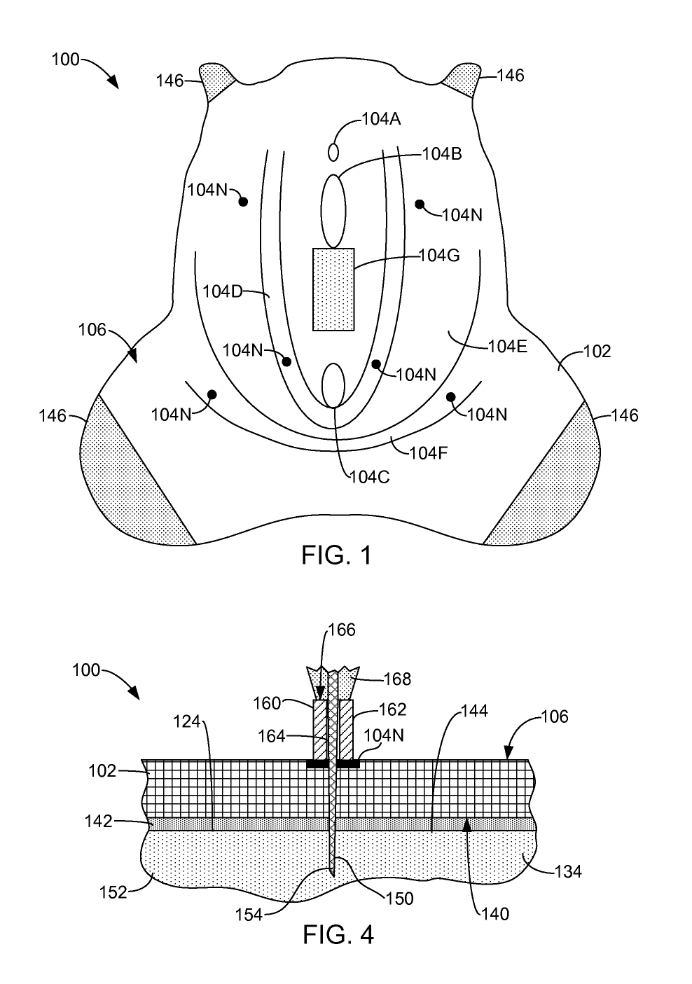

FIG. 1 is a simplified top view of a perineum overlay device 100 in accordance with embodiments of the invention. In some embodiments, the device 100 is designed to assist in locating or identifying anatomical features within the perineum of a patient and/or needle insertion sites for targeting anatomical features within the perineum of a patient, to simplify pelvic condition treatments. The pelvic condition treatments that may be simplified using the device 100 include treatments that are designed to alleviate pelvic floor muscle hypertonicity and/or spasm, which may be effective in treating other pelvic disorders rooted from pelvic floor neuromuscular abnormalities. Such treatments may involve the injection of drugs, such as botulinum toxin (Botox), recording electromyogram signals from muscles of the pelvic floor, electrically stimulating muscles of the pelvic floor, and/or implanting an electrode in muscles of the pelvic floor that may be used to perform an electrical stimulation therapy, for example. The device 100 may be useful in other pelvic condition treatments.

In some embodiments, the device 100 includes a section of flexible material 102 that is configured to conform and cover a portion of the perineum of a patient. A plurality of markings, generally referred to as 104, are printed on a first side 106 of the section 102. Each of the markings 104 is configured to identify a location of an anatomical feature within the perineum, and/or a needle insertion site corresponding to a targeted anatomical feature within the perineum, when the section of flexible material 102 is positioned over the perineum of a patient.

In some embodiments, the locations of the markings 104 on the section of flexible material 102 are determined based on a patient model, such as an average female patient. Devices 100 may be designed based on other patient models in order to accommodate a range of individuals. As a result, a device 100 may be selected for a patient such that the markings 104 align with the corresponding anatomical features of the patient, when the device 100 is placed over the perineum of an actual patient.

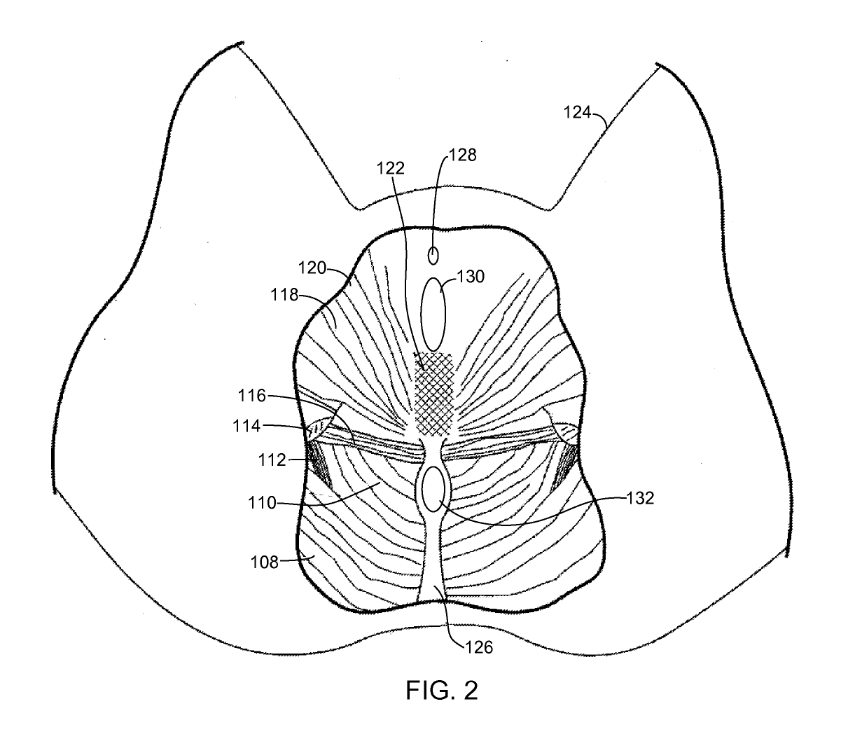

Exemplary anatomical features of the perineum that may be identified or targeted by the markings 104 will be described with reference to FIG. 2, which illustrates a view of inferior tissue at different levels of a pelvic region, including the gluteus maximus 108, the levator ani 110 (which includes the iliococcygeus muscle), sacrotuberous ligament 112, ischial tuberosity 114, superficial transverse perineal muscle 116, pubococcygeus muscle 118, puborectalis muscle 120, and the perineal body 122. The epidermis 124, the coccyx 126, the urethra 128, the vagina 130, and the anus 132 are also shown in FIG. 2.

Thus, in some embodiments, the device 100 includes an anatomical marking 104A corresponding to the urethra of a patient model, an anatomical marking 104B corresponding to the vagina of the patient model, an anatomical marking 104C corresponding to the anus of the patient model, an anatomical marking 104D corresponding to the puborectalis muscle of the patient model, an anatomical marking 104E corresponding to the pubococcygeus muscle of the patient model, an anatomical marking 104F corresponding to the iliococcygeus muscle of the patient model, 104G corresponding to the perineal body of the patient model, and/or other muscle or anatomical feature beneath the perineum of the patient model.

In some embodiments, the markings 104 comprise graphical markings that are used to identify anatomical features. Such graphical markings may comprise various shapes, lines, colors, shading, and other graphical features to identify the anatomical feature to which the markings 104 correspond.

In some embodiments, the markings 104 include textual markings or labels that identify the anatomical feature to which they correspond. For instance, a marking 104 may include the name of the anatomical feature written out, or a reference number or character from which the corresponding anatomical feature may be identified using a look-up table, for example.

In some embodiments, the markings 104 include openings in the section of flexible material 102. These openings allow the physician to observe the perineum of the patient through the section of flexible material 102. In some embodiments, the openings of the markings 104 are configured to be placed over the corresponding anatomical feature of the patient.

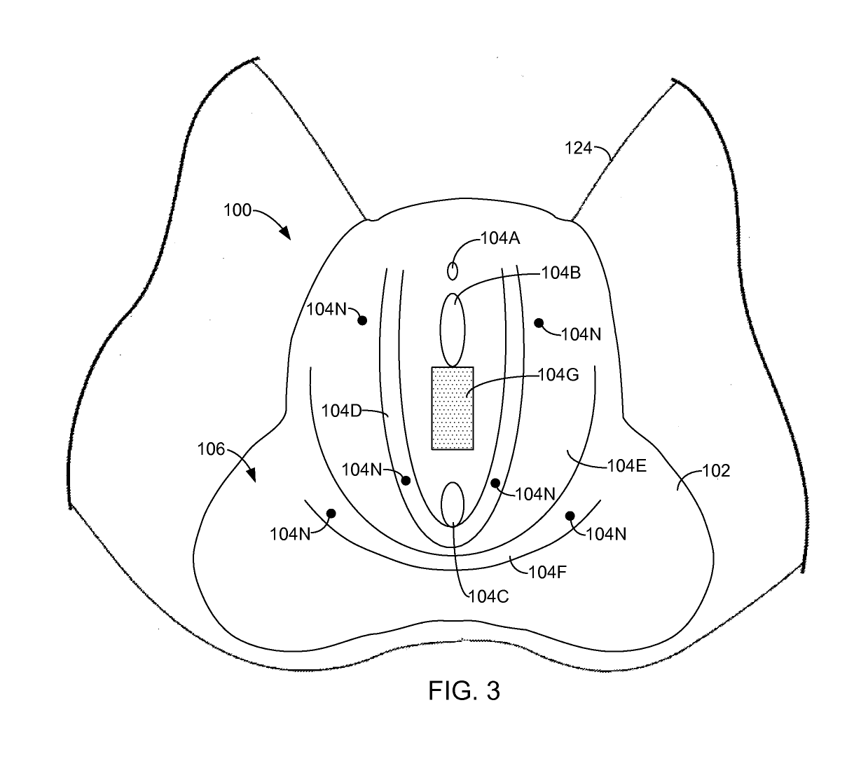

In some embodiments, the perineal overlay device 100 is configured to be placed over the perineum of a patient, as shown in the simplified illustration of FIG. 3. In some embodiments, the device 100 is positioned over the perineum such that the markings 104 identifying anatomical features directly overlay the corresponding anatomical features (FIG. 2) of the patient. Thus, when the perineum overlay device 100 is properly positioned over the perineum of the patient, a marking 104A corresponding to the urethra of a patient overlays the urethra 128 of the patient, a marking 104B corresponding to the vagina should overlay the vagina 130 of the patient, a marking 104C corresponding to the anus of the patient should overlay the anus 132 of the patient, a marking 104D corresponding to the puborectalis muscle, should overlay the puborectalis muscle 210 of the patient, an anatomical marking 104E corresponding to the pubococcygeus muscle should overlay the pubococcygeus muscle 118 of the patient, an anatomical marking 104F corresponding to the iliococcygeus muscle should overlay the iliococcygeus muscle (levator ani 110) of the patient, and so forth.

In some embodiments, some of the markings 104 are configured as alignment markings to assist the physician in aligning the markings 104 of the perineal overlay device 100 to the corresponding anatomical features of the patient 134. The alignment markings 104 may include some of the markings 104 described above, such as the markings corresponding to the urethra (104A), the vagina (104B), and the anus (104C), for example. These markings may have openings through which the physician can locate the corresponding anatomical feature of the patient to assist in aligning the device 100. In some embodiments, a physician may mark a location on the epidermis of the patient corresponding to an anatomical feature of the patient 134, which may then be aligned to a marking 104 on the device 100 to assist in aligning the device 100 to the patient 134.

In some embodiments, a back side 140 of the section of flexible material 102 comprises an adhesive layer 142, as illustrated in the cross-sectional view of the device 100 overlaying the perineum 144 of the patient 134 provided in FIG. 4. The adhesive layer 142 is preferably configured to bond to the epidermis 124 of the patient and prevent shifting of the device 100 relative to the perineum of the patient 144. In some embodiments, the adhesive layer 142 is located only at select portions of the section of flexible material 102, such as the shaded sections 146 illustrated in FIG. 1. Alternatively, the physician may apply a suitable adhesive to the epidermis 124 of the patient to adhere one or more portions of the material 102 to the patient.

After one or more of the markings 104 have been aligned with the corresponding anatomical features of the patient 134, the device 100 may be used to assist the physician in performing a pelvic treatment configured to treat a pelvic condition of the patient, such as those mentioned above. In some embodiments, the pelvic treatment involves the insertion of a needle 150 into tissue 152 within the perineum 144 of the patient, as illustrated in FIG. 4. The needle 150 may be in the form of an electrode needle, an introducer needle, or a needle configured to inject a substance into the tissue 152. In some embodiments, the pelvic treatment involves the positioning of the needle 150 within a specific anatomical feature of the patient, such as a muscle within the perineum 144. In some embodiments, the perineum overlay device 100 is used to assist in determining the location where the needle 150 is to be inserted in order to perform the desired pelvic treatment.

In some embodiments, the physician may use the anatomical markings 104 to locate the desired anatomical feature of the patient and identify the location where the needle 150 is to be inserted. In some embodiments, the markings 104 include one or more needle insertion site markings 104N that identify specific locations where a needle 150 is to be inserted to perform a pelvic treatment. The physician identifies the needle insertion site marking 104N corresponding to the desired pelvic treatment to be performed, and inserts the appropriate needle 150 through the needle insertion marking 104N and performs the treatment using the needle 150, as shown in FIG. 4.

In some embodiments, the pelvic treatment requires the needle 150 to be positioned at a certain depth in order to reach the targeted anatomical feature on which the pelvic treatment is to be performed. For instance, the anatomical feature may comprise a muscle within the perineum 144 of the patient 134 that is at a known depth within the tissue relative to the corresponding needle insertion site marking 104N when the device 104 is properly positioned over the perineum 144 of the patient. In some embodiments, the physician selects an appropriate length needle 150 that positions the distal end 154 at the desired depth within the tissue 152 of the patient.

In some embodiments, the device 100 includes one or more needle stops 160, a cross-sectional view of which is provided in FIG. 4. Each needle stop 160 is attached to the side 106 of the section of flexible material 102 where a pelvic treatment is to occur, and assists in controlling the depth at which the needle 150 is inserted into the patient. In some embodiments, the one or more needle stops 160 are positioned at needle insertion site markings 104N, as shown in FIG. 4, or operate as needle insertion site markings themselves.

In some embodiments, the needle stop 160 comprises a body 162 having a cylindrical bore 164, through which the needle 150 may be inserted. In some embodiments, the needle stop 160 includes a shoulder portion 166 that is configured to engage a portion 168 of the needle 150 to prevent further movement of the needle 150 through the bore 164 and into the patient. Thus, the depth at which the needle 150 may be inserted into the patient 134 is limited by the needle stop 160. This allows for single sized needles to be used by the physician to perform pelvic treatments while using needle stops 160 having various sizes to ensure that the needles are inserted in the patient at appropriate depths depending on the location of the needle insertion site.

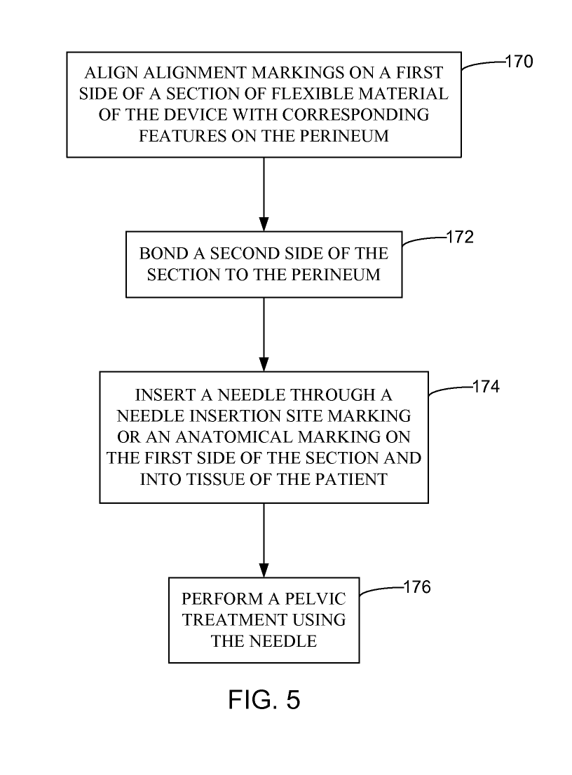

Some embodiments of the invention are directed to a method of using the perineal overlay device 100, formed in accordance with one or more embodiments described above, to perform a pelvic treatment on a patient 134. FIG. 5 is a flowchart illustrating a method of treating a pelvic condition of a patient in accordance with embodiments of the invention. At 170 of the method, one or more alignment markings on a first side 106 of a section of flexible material 102 of a perineal overlay device 100 are aligned with corresponding features on the perineum of a patient. As mentioned above, the alignment markings may comprise graphical or textual markings, or openings in the section of flexible material 102. These alignment markings may be aligned with anatomical features of the patient, such as the urethra 128, the vagina 130, the anus 132, and/or other anatomical features of the patient. Additionally, the alignment markings may be aligned with markings applied to the epidermis 124 of the patient.

At 172, a second side 140 of the section of flexible material 102 is bonded to the perineum 144 of the patient 134 while the device 100 is properly aligned, as shown in FIG. 3. In some embodiments, one or more portions of the second side 140 of the section of flexible material 102, such as portions 146 (FIG. 1), include an adhesive layer 142 that is configured to bond the section of flexible material 102 to the skin 124 of the patient to bond the device 100 in the desired alignment with the perineum of the patient. In other embodiments, a physician may apply an adhesive to the epidermis 124 of the patient to bond the perineal overlay device 100 to the perineum 144 of the patient.

At 174, a needle 150 is inserted through a needle insertion site marking 104N or an anatomical marking on the first side 106 of the section of flexible material 102 and into tissue 152 of the patient 134, as shown in FIG. 4. As discussed above, the needle insertion site marking 104N is aligned with an anatomical feature of the patient, such as a muscle within the perineum 144 that is targeted for a pelvic treatment. Alternatively, the physician may determine an appropriate location to insert the needle 150 based on the anatomical markings 104 provided on the section of flexible material 102, such as those described above. In some embodiments, a needle stop 160 is used to assist in the insertion of the needle 150 into the patient, as described above.

At 176, a pelvic treatment is performed on the patient 134 using the needle 150. Embodiments of the pelvic treatment include injecting a substance (e.g., Botox) into the targeted tissue 152, recording electromyogram signals produced by the targeted tissue 152, electrically stimulating the targeted tissue 152, and/or implanting an electrode in the targeted tissue 152. Other pelvic condition treatments may also be performed.

Although the present invention has been described with reference to preferred embodiments, workers skilled in the art will recognize that changes may be made in form and detail without departing from the spirit and scope of the invention.

1. A perineum overlay device for locating anatomical features within the perineum of a patient and/or needle insertion sites for targeting anatomical features within the perineum of a patient, the device comprising:
a section of flexible material configured to conform and cover a portion of the perineum of a patient; and
a plurality of markings printed on a first side of the section, each marking configured to identify a location of at least one of an anatomical feature within the perineum and a needle insertion site corresponding to a targeted anatomical feature within the perineum, when the section is positioned over the perineum of a patient.

2. The device of claim 1, further comprising an adhesive layer on a second side of the section that is opposite the first side.

3. The device in accordance with claim 1, wherein the section is configured to cover the vagina of a patient.

4. The device in accordance with claim 1, wherein the section is configured to cover the urethra of a patient.

5. The device in accordance with claim 1, wherein the section is configured to cover the anus of a patient.

6. The device in accordance with claim 1, wherein the markings include at least one anatomical marking configured to identify a location of one or more muscles selected from the group consisting of the puborectalis muscle, the pubococcygeus muscle, and the iliococcygeus muscle.

7. The device in accordance with claim 1, wherein the markings include at least one anatomical marking configured to identify a location of an anatomical feature of a patient selected from the group consisting of the vagina, the urethra, and the anus.

8. The device in accordance with claim 1, wherein the markings include at least one needle insertion site marking configured to identify a needle insertion site.

9. The device in accordance with claim 1, wherein the markings comprise graphical markings.

10. The device in accordance with claim 1, wherein the markings comprise textual markings.

11. The device in accordance with claim 1, wherein the markings comprise one or more openings in the section of flexible material.

12. The device in accordance with claim 1, further comprising a needle stop attached to the first side of the section, the needle stop including a body having cylindrical bore through which a needle may be inserted.

13. The device of claim 12, wherein the needle stop is positioned at a needle insertion site.

14. The device of claim 13, wherein the needle stop is positioned over one of the markings identifying a needle insertion site.

15. A method of treating a pelvic condition of a patient comprising:
positioning a perineum overlay device comprising a section of flexible material over the perineum of a patient comprising:
aligning alignment markings on a first side of the section with corresponding features on the perineum; and
bonding a second side of the section to the perineum;
inserting a needle through a needle insertion site marking or an anatomical marking on the first side of the section and into tissue of the patient; and
performing a pelvic treatment using the needle.

16. The method of claim 15, wherein:
the alignment markings comprise at least one anatomical marking identifying an anatomical feature of the patient selected from the group consisting of the vagina, the urethra, and the anus; and
aligning alignment markings comprises aligning the at least one anatomical marking with the corresponding anatomical feature of the patient.

17. The method in accordance with claim 16, wherein:
the alignment markings comprise at least one opening; and
aligning alignment markings comprises aligning the at least one opening to a mark applied to the epidermis of the perineum of the patient or an anatomical feature of the patient.

18. The method in accordance with claim 15, wherein bonding a second side of the section to the perineum comprises bonding an adhesive layer attached to the second side of the section to epidermis of the perineum.

19. The method in accordance with claim 15, wherein inserting a needle through a needle insertion site marking or an anatomical marking on the first side of the section and into tissue of the patient comprises:
inserting a needle through a cylindrical bore of a needle stop attached to the first side of the section; and
limiting a depth at which the needle is inserted into the tissue through engagement with a shoulder portion of the needle stop.

20. The method in accordance with claim 15, wherein performing a pelvic treatment using the needle comprises at least one treatment selected from the group consisting of injecting a substance into the tissue, recording electromyogram signals, electrically stimulating the tissue, and implanting an electrode in the tissue.
